(19) United States
(12) Patent Application Publication
(10) Pub. No.: US 2008/0112944 A1
(43) Pub. Date: May 15, 2008

(54) COMPOSITIONS AND METHODS FOR THE TREATMENT OF AUTISM

(75) Inventors: Jon B. PANGBORN, St. Charles, IL (US); Larry NEWMAN, Portland, OR (US); Rohit MEDHEKAR, Springfield, MO (US); Anthony COLLIER, Forsyth, MO (US); Steven MARR, Forsyth, MO (US)

Correspondence Address: SPECKMAN LAW GROUP PLLC, 1201 THIRD AVENUE, SUITE 330, SEATTLE, WA, 98101, US

(73) Assignees: KIRKMAN GROUP, INC., Laked Oswego, OR (US); NATIONAL ENZYME COMPANY, INC., Forsyth, MO (US)

(21) Appl. No.: 12/015,444

(22) Filed: Jan. 16, 2008

Related U.S. Application Data
(62) Division of application No. 11/000,668, filed on Dec. 1, 2004.

(51) Int. Cl. A61K 38/54; A61K 38/47

(52) U.S. Cl. 424/94.2; 424/94.61

(57) ABSTRACT

Compositions that may be usefully employed to alleviate symptoms resulting from deficiencies in carbohydrate enzymes, together with methods for the treatment of disorders that are characterized by such deficiencies, such as autism, are provided. The compositions preferably comprise transglucosidase isolated from A. niger.

### REFERENCE TO RELATED APPLICATIONS

This application is a divisional of U.S. patent application Ser. No. 11/000,668, filed Dec. 1, 2004.

### TECHNICAL FIELD OF THE INVENTION

The present invention relates to methods and compositions for the treatment of autism and other disorders characterized by a deficiency in one or more digestive enzymes. More specifically, the present invention relates to the treatment of such disorders by the administration of nutritional supplements that aid digestion.

### BACKGROUND OF THE INVENTION

Autism (also referred to as Autism Spectrum Disorder, or ASD) is a disorder that seriously impairs the functioning of individuals. It is characterized by self-absorption, a reduced ability to communicate with or respond to the outside world, rituals and compulsive phenomena, and mental retardation. Autistic individuals are also at increased risk of developing seizure disorders, such as epilepsy. Autism, which is generally diagnosed by age three, is about two to five times more common in boys than girls, and its incidence appears to be increasing. While the actual cause of autism is unknown, it appears to include one or more genetic factors, as indicated by the fact that the concordance rate is higher in monozygotic twins than in dizygotic twins, and may also involve immune and environmental factors, such as diet, toxic chemicals and infections.

The human intestinal tract contains seven enzymes which split dietary disaccharides into free monosaccharides:
trehalase (EC 3.2.1.28) which acts on the sugar trehalose that comes from fungi and yeast;
lactase (EC 3.2.1.23) which acts on lactose; glucosylceramidase (EC 3.2.1.45 and 46); and phlorizin hydrolase (EC 3.2.1.62), which are all contained with the beta-glucosidase complex;
glycoamylase complex (EC 3.2.1.20; also known as glycoamylase 1 plus glycoamylase 2, or heat-stable maltase 1 plus heat-stable maltase 2); and
sucrase (EC 3.2.1.48; also called heat-labile maltase) and isomaltase (EC 3.2.1.10), which are both contained with the sucrase-isomaltase complex.

Prior to the late 1990's, autism was believed to possibly feature only incomplete digestion of protein, not carbohydrates. In the early 1990's, analysis of the urine of autistic children demonstrated significantly increased levels of peptides, in particular the exorphins casomorphin and gluteomorphin, compared to normal individuals (Reichelt et al. J. Applied Nutr., 42:1-11 (1990)). Casomorphins are formed during the digestion and metabolism of casein, a primary protein in milk products, while gluteomorphins are formed during the digestion and metabolism of gluten, a primary protein of wheat products. These exorphins have been shown to have opiate-type effects on the body and have been implicated in a variety of human diseases including schizophrenia and attention deficit disorder. More specifically, opioid peptides can stimulate T cells, and induce peptide specific T cell responses and abnormal levels of cytokine production, which in turn can lead to inflammation, autoimmune reactions and disruption of neuroimmune communications. It has been shown that eliminating gluten and casein from the diet by following a strict wheat and dairy-free diet, greatly improves the symptoms of autistic children. However, complete elimination of gluten and casein from the diet is difficult to achieve and hence there has been a great deal of interest in nutritional supplements that improve the digestion of protein in autistic individuals (see, for example U.S. Pat. Nos. 6,251,391 and 6,783,757).

In 1999, Horvath et al. published findings of carbohydrate maldigestion in autistics (J. Pediatrics, 135:559-563, 1999). In a clinical study of 36 autistic children, 58% were found to have subnormal carbohydrate digestive enzyme activity. Horvath et al. determined that disaccharidases and/or glycoamylase were at fault. In a subsequent study on 112 autistic patients, Horvath and Perman found that over half of the patients had symptoms consistent with maldigestion and again provided evidence of carbohydrate maldigestion (Horavth and Perman Curr Opin. In Pediatrics 14:583-587 (2002)). In particular, they identified deficiencies in lactase, maltase, sucrase, palatinase and glucoamylase in 58% of the patients. Palatinase (also known as isomaltase) is of particular interest as it is expressed in the same crypts in the intestinal mucosa as dipeptidylpeptidase 4 (DPP4), which is the peptidase responsible for digesting many exorphin peptides (Gorvel et al., Gastroenterology 101:618-625 (1991); Misumi and Ikahara, in Handbook of Proteolytic Enzymes, ed. Barrett, Rawlings and Woessner, Academic Press, p. 387-382 (1998)).

More recently, Kushak and Buie of Massachusetts General Children's Hospital reported on intestinal biopsy findings of over 100 autistic individuals, in which they found that 60-65% of the individuals had weak lactase activity and 25-45% had weak isomaltase/palatinase activity, indicating that many autistic individuals are deficient in these enzymes. However, food grade isomaltase is not commercially available in large quantities and is thus not readily available for use as a digestive aid. There thus remains a need for a readily available nutritional supplement that would be beneficial for individuals suffering from a deficiency in isomaltase and other digestive enzymes, such as autistic patients.

### SUMMARY OF THE INVENTION

The present invention provides compositions that may be usefully employed to alleviate symptoms resulting from deficiencies in carbohydrate-digesting enzymes, together with methods for the treatment of disorders that are characterized by such deficiencies. Disorders that may be treated using the inventive compositions include, but are not limited to, autism (also referred to as autistic spectrum disorder, or ASD), inflammatory bowel disease, Crohn's disease, irritable bowel syndrome and ulcerative colitis.

As detailed below, the inventors have determined that transglucosidase (in particular transglucosidase from Aspergillus niger) may be usefully employed to compensate for a deficiency in the enzyme isomaltase (also known as palatinase), and may therefore be employed to treatment autism. The compositions of the present invention thus comprise transglucosidase, preferably isolated from A. niger. Other sources of transglucosidase which may be usefully employed in the inventive compositions includes molds, bacteria and yeast. The inventive compositions may also contain one or more additional components believed to be useful in the treatment of disorders characterized by a deficiency in other carbohydrate-digesting enzymes. Preferably, such components are selected from the group consisting of: glucoamylase, lactase, invertase, amylase, maltase and malt diastase.

In certain embodiments, the compositions of the present invention additionally comprise one or more components believed to be beneficial in the treatment of disorders characterized by incomplete digestion of proteins, lipids and/or other non-carbohydrate materials commonly present in foods. Preferably, such components are selected from the group consisting of: peptidases, proteases, cysteine proteases (such as bromelain and papain), phytase, α-galactosidase, cellulase, xylanase, lipase, and combinations thereof.

In other aspects, the present invention provides methods for the treatment of a disorder selected from the group consisting of: autism; inflammatory bowel disease; Crohn's disease; irritable bowel syndrome; and ulcerative colitis, such methods comprising administering one or more of the inventive compositions. Preferably the compositions are formulated in a tablet or capsule form and are taken with meals.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 shows the effect of increasing concentrations of A. niger transglucosidase on the release of glucose from isomaltose.

FIG. 2 shows the amount of glucose liberated from palatinose by a fixed concentration of transglucosidase over time.

FIG. 3 shows the amount of glucose liberated from various concentrations of palatinose by a fixed concentration of transglucosidase.

### DETAILED DESCRIPTION OF THE INVENTION

As discussed above, the present invention provides compositions formulated to overcome deficiencies in carbohydrate-digesting enzymes that have been identified in patients with autism. In preferred embodiments, the compositions include a component that is believed to overcome deficiencies in the enzyme isomaltase, together with components that are believed to overcome deficiencies in one or more enzymes selected from the group consisting of: lactase, maltase, sucrase, amylase and glucoamylase.

Maltose is a disaccharide sugar composed of one molecule of glucose joined to another molecule of glucose by a 1→4 glycosidic bond. During digestion, this bond is broken by the enzyme maltase (also known as α-glucosidase; EC 3.1.1.20) to yield two molecules of glucose. Isomaltose is a disaccharide sugar composed of one molecule of glucose joined to another molecule of glucose by a 1→6 glycosidic bond. This 1→6 glycosidic bond is broken during digestion by the enzyme isomaltase (EC3.2.1.10; dextrin-6-α-D-glucanohydrolase) to give two molecules of glucose. Maltase cannot substitute for isomaltase. Palatinose (occasionally referred to as isomaltulose) is a disaccharide sugar composed of one molecule of glucose joined to one molecule of fructose (fructofuranose) by a 1→6 glycosidic bond. Isomaltase also breaks this 1→6 bond to produce one molecule of glucose and one molecule of fructose.

Transglucosidase (EC 2.4.1.24) is an alpha-glucosidase extracted from culture broths of the fungal plant Aspergillus niger. It is a food grade enzyme that is used in grain processing and brewing, and is known to have some isomaltase activity (McCleary et al. Carbohydrate Research 185:147-162 (1989)). However, explicit activity in palatinose digestion has not been previously documented and, prior to its use in nutritional supplements, the ability of transglucosidase to promote undesirable reverse reactions had to be ruled out by testing as detailed below in Example 1.

Lactase (also known as β-galactosidase; EC 3.2.1.23) is a disaccharidase that cleaves lactose (milk sugar) into its component sugars fructose and galactose. The inclusion of lactase in the inventive compositions permits utilization of the compositions by lactose intolerant people and increases the amount of available galactose.

Invertase (EC 3.2.1.26; obtained from yeast) is a disaccharidase that acts on sucrose to yield glucose and fructose, and that hydrolyzes other complex sugars that contain fructose as a β-D-fructofuranoside. It is used in digestive aid supplements in place of the enzyme sucrase, as actual food-grade analogs of human sucrase are not commercially available.

Amylase (obtained from vegetable pancreatin) and glucoamylase (EC 3.2.1.3; isolated from A. niger) are enzymes that break starch down into smaller polysaccharides, disaccharides or glucose itself Malt diastase is characterized by its ability to hydrolyze amylose and other polysaccharides. This enzyme works synergistically with amylase and glucoamylase to digest carbohydrate rich foods, particularly those produced from grains.

In addition to containing components that overcome deficiencies in carbohydrate-digesting enzymes, the inventive compositions may also include components that overcome deficiencies in other digestive enzymes, such as enzymes important in the digestion of proteins and/or lipids. In certain embodiments, such compositions comprise at least one component selected from the group consisting of: peptidases; proteases; cysteine proteases, such as bromelain; phytases; α-galactosidase; cellulase; lipase; and xylanase.

In one embodiment, a peptidase concentrate component is included that exhibits both endo- and exo-peptidase activity. In an alternative embodiment, the peptidase concentrate included in the inventive composition mimics dipeptidyl-peptidase IV (DPPIV; EC 3.4.14.5) activity and hence provides further exorphin digestion (see, for example, U.S. Pat. No. 6,783,757).

The inventive compositions preferably comprise at least one protease that has high acid and/or alkaline stability and functions in the stomach to hydrolyze large proteins into smaller peptides. Such proteases are preferably isolated from plants, such as kiwi. An example of an acid stable protease component that may be included in the inventive composition is Protease 3.0, available from National Enzyme Company (Forsyth, Mo.). Another example of a protease component that may be usefully employed in the inventive compositions is Protease 6.0, also available from National Enzyme Company, which is a mixture of acid, neutral and alkaline proteases that demonstrates both exo-peptidase and endo-peptidase activity with high substrate specificity.

To further assist with protein digestion, the inventive compositions preferably comprise a cysteine protease. Bromelain and papain are examples of cysteine proteases which may be effectively employed in the compositions. Bromelain is preferred over papain as it is believed that bromelain has a wider specificity and function than papain. It has also been demonstrated that bromelain is an effective anti-inflammatory, which may be significant in reducing the “leaky gut” characteristic of autistic individuals.

Phytase is preferably added for its ability to digest phytic acid, which is present in plants such as corn, rice, wheat, soybean and other beans. Phytic acid can negatively affect absorption of minerals such as zinc, calcium, magnesium, copper, manganese and iron. The inclusion of phytase thus results in greater bioavailability of these minerals.

α-Galactosidase is characterized by its ability to hydrolyze the alpha-1-6 linkages in melibiose, raffinose, and stachyose, which are commonly found in vegetables and legumes. These sugars are not readily digested by humans and can cause considerable digestive discomfort. The inclusion of this enzyme therefore reduces digestive discomfort and provides a source of nutrition not normally available to humans.

Xylanase hydrolyzes xylans, which are indigestible components of plant fibers. Since humans lack the endogenous enzymes required to digest plant fibers, the inclusion of xylanase provides an additional source of nutrition. Similarly, the inventive compositions preferably include cellulase in order to improve the digestion of cellulose present in plant foods.

The components included in the inventive compositions are readily available commercially. They are preferably provided in a dry form, then mixed and encapsulated to provide a formulation suitable for oral delivery. The resulting capsules or tablets are preferably taken with food. One of skill in the art will appreciate that other delivery methods may be utilized without departing from the present invention. The specific concentrations of components included in the inventive compositions can vary, but generally correspond to those currently employed in commercially available nutritional supplements. Additional, inactive, components may be included such as, but not limited to, microcrystalline cellulose, magnesium stearate, silicon dioxide, rice bran and mineral oil.

In a first preferred embodiment (referred to as Formulation I), each capsule contains the following active ingredients:

| | | |
|---|---|---|
| Glucoamylase | 100 | AGU |
| A. niger transglucosidase | 100 | mg |
| Malt diastase | 800 | DP |
| Lactase | 2000 | ALU |
| Invertase | 1000 | SU |
| Amylase | 200 | DU |

Wherein, AGU = Amyloglucosidase Units, DP = Diastatic Power, SU = Sumner Units, DU = Dextrinizing Units, ALU = Lactase Units (also known as LAU).

In a second preferred embodiment (referred to as Formulation II), each capsule contains the following active ingredients:

| | | |
|---|---|---|
| Peptidase | 2500 | HUT |
| A. niger transglucosidase | 50 | mg |
| Protease 3.0 | 50 | SAPU |
| Bromelain | 640,000 | FCCPU |
| Papain | 1,000,000 | FCCPU |
| α-Galactosidase | 25 | GalU |
| Invertase | 200 | SU |
| Cellulase | 100 | CU |
| Xylanase | 50 | XU |
| Amylase | 50 | DU |
| Protease 6.0 (conc) | 875 | HUT |
| Malt diastase | 13 | DP |
| n*zimesPA ™* | 55 | mg |
| n*zimes ™* | 269 | mg |

(*proprietary blends of lipase, protease and amylase available from the National Enzyme Co. (Forsyth, MO)) wherein, HUT = Hemoglobin Units Tyrosine, SAPU = Spectrophotometric Acid Protease Units, FCCPU = Food Chemical Codex Papain Units, GalU = Galactosidase Unit (also known as AGSU), CU = Cellulase Units, and XU = Xylanase Units

The preferred dosage for each of these formulations is one to two capsules (in the case of Formulation II, 50 or 100 mg of transglucosidase) taken with meals, with the dosage varying with the size of the meal and/or the body weight of the patient. For young children, half a capsule may be taken with each meal.

The following Examples are offered by way of illustration and not by way of limitation.

### EXAMPLE 1

### Determination of Activity of Transglucosidase In Vitro

It is known that A. niger transglucosidase has some isomaltase activity. However, in order for transglucosidase to be appropriate for treatment of isomaltase deficiency in, for example, autistic individuals, it must have the following functional properties:
(a) it must be able to split one molecule of isomaltose into two molecules of glucose;
(b) it must be able to split one molecule of palatinose into one molecule of glucose and one molecule of fructose;
(c) it must not activate the reverse of either (a) or (b) when only glucose, or glucose and fructose, are present; and
(d) it must not convert maltose to isomaltose.

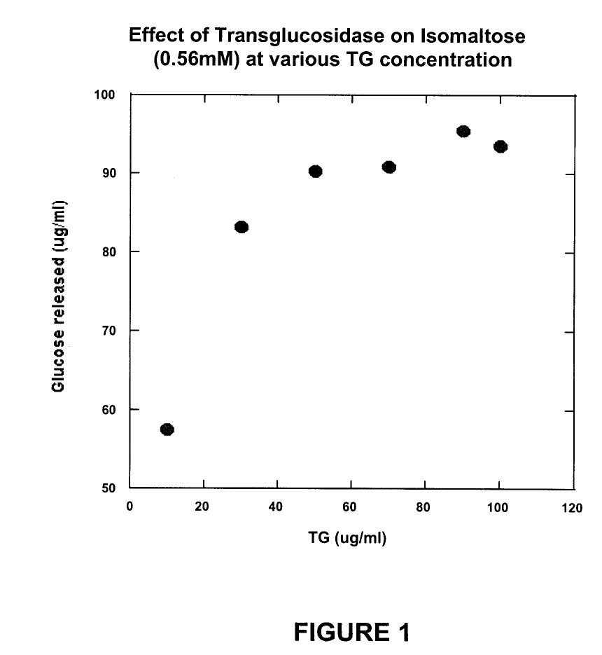

In order to test these properties, various amounts of A. niger transglucosidase (TG) were reacted with isomaltose in a broth at near body temperature to confirm that isomaltose is indeed converted to glucose and to determine what concentrations of transglucosidase are needed for the conversion. The results of this study are shown in FIG. 1. These results indicate that TG does convert isomaltose to glucose, with concentrations equal to or greater than 40 μg/ml being required. Concentrations above 70 μg/ml were found to provide little additional benefit. Almost 100% conversion of isomaltose to glucose was observed. These results indicate that concentrations of TG between 40-70 μg/ml are optimal for this conversion.

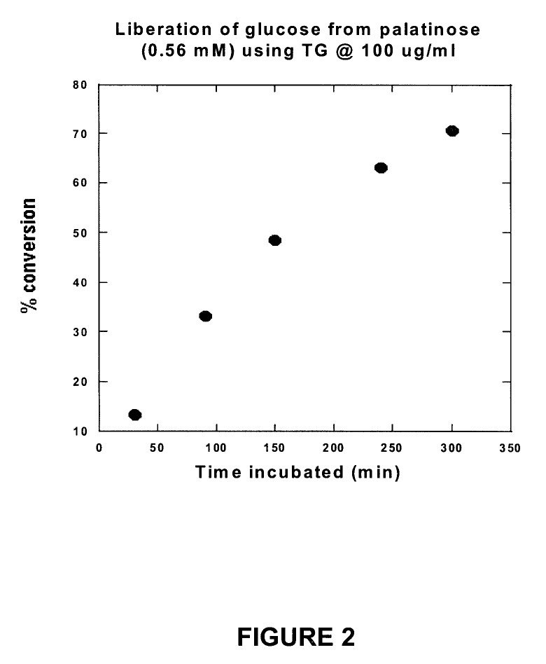

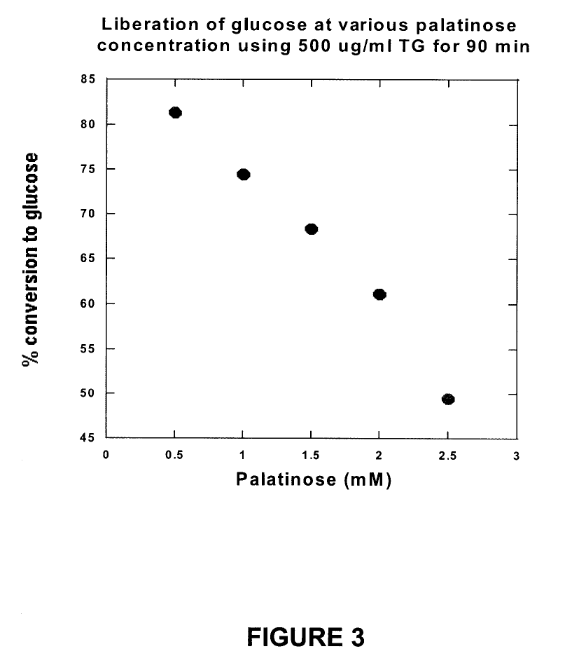

The ability of TG to convert palatinose to glucose and fructose was examined by measuring the release of glucose from a broth containing 100 μg/ml TG. It is known that more TG is needed for conversion of palatinose than for conversion of isomaltose. FIG. 2 shows the liberation of glucose from palatinose (measured as the percentage conversion to glucose) over time by TG at a concentration of 100 μg/ml. FIG. 3 shows the percentage conversion of palatinose to glucose after 90 minutes with varying concentrations of TG. TG was found to convert palatinose to glucose and fructose, although conversion was slower than for isomaltose to glucose, with just over 50% conversion being achieved in 180 minutes.

In experiments testing the conversion of glucose back to isomaltose with TG, no loss of glucose was found from the test broth, indicating that there was no formation of isomaltose, maltose or any other complex sugar. In studies testing the conversion of maltose to isomaltose with TG, at the concentrations of TG tested (up to 120 μg/ml), no conversion of maltose to isomaltose could be detected.

Based on the above tests, it was determined that A. niger transglucosidase, EC 2.4.1.24, qualifies qualitatively as a substitute enzyme for isomaltase, EC 3.2.1.10. As palatinose is a very minor disaccharide component of fruits and vegetables, further tests were performed to determine how conversion of palatinose varies with its concentration. Conversions were determined to be concentration-dependent, with the less palatinose, the higher the conversion to glucose and fructose for given concentrations of TG and incubation times.

These studies indicate that A. niger transglucosidase has satisfactory activity as a digestive enzyme for isomaltose as determined by in vitro testing. While A niger TG has less activity for palatinose, palatinose is a very minor sugar in carbohydrate foods, and thus dietary supplementation with TG may be satisfactory even though conversion rates are slower.

### EXAMPLE 2

### Activity of Compositions Containing Transglucosidase In Vivo

In order to assess the effectiveness of the inventive compositions in the treatment of autism, individuals previously diagnosed with autism were provided with capsules of either Formulation I or Formulation II, as described above, or both Formulation I and Formulation II, and instructed to take 1-2 capsules with each meal. Patients and/or their doctors were requested to provide information regarding changes in gastrointestinal discomfort, overall tolerance to foods, stimming, hyperactivity, mood, attention, sleep, eye contact, speech, socialization and compulsions, together with information regarding any undesirable side effects or sensitivity type reactions, such as allergic reactions or rashes.

Initial results indicate that the Formulations are well tolerated by patients, with almost no adverse reactions. In addition, encouraging preliminary reports on benefit have been received from patients.

From the foregoing it will be appreciated that, although specific embodiments of the invention have been described herein for purposes of illustration, various modifications may be made without deviating from the spirit and scope of the invention.

We claim:

1. A method for the treatment of a disorder characterized by a deficiency of isomaltase in a subject, comprising administering to the subject a composition comprising transglucosidase.

2. The method of claim 1, wherein the transglucosidase is from Aspergillus niger.

3. The method of claim 1, wherein the disorder characterized by a deficiency of isomaltase is selected from the group consisting of: autism; inflammatory bowel disease; Crohn's disease; irritable bowel syndrome; and ulcerative colitis.

4. The method of claim 1, wherein the composition further comprises at least one additional carbohydrate-digesting enzyme.

5. The method of claim 4, wherein the at least one additional carbohydrate-digesting enzyme is selected from the group consisting of: glucoamylase (EC 3.2.1.3); maltase;
malt diastase; lactase (EC 3.2.1.23); invertase (EC 3.2.1.26); and amylase.

6. The method of claim 1, wherein the composition further comprises at least one non-carbohydrate-digesting enzyme.

7. The method of claim 6, wherein the non-carbohydrate-digesting enzyme is selected from the group consisting of: peptidases; proteases; cysteine proteases; phytases; α-galactosidase; cellulase; lipase; and xylanase.

8. The method of claim 7, wherein the peptidase is dipeptidylpeptidase IV.

9. The method of claim 7, wherein the cysteine protease is selected from the group consisting of: bromelain and papain.

10. A method for the treatment of a disorder characterized by a deficiency of isomaltase in a subject, comprising administering to the subject a composition comprising:
(a) transglucosidase;
(b) at least component selected from the group consisting of: glucoamylase (EC 3.2.1.3); maltase; malt diastase; lactase (EC 3.2.1.23); invertase (EC 3.2.1.26); and amylase; and
(c) at least one component selected from the group consisting of: peptidases; proteases; cysteine proteases; phytases; α-galactosidase; cellulase; lipase; and xylanase.

11. The method of claim 10, wherein the composition comprises: transglucosidase, glucoamylase (EC 3.2.1.3), malt diastase, lactase (EC 3.2.1.23), invertase (EC 3.2.1.26), amylase, a peptidase, a protease, a cysteine protease, α-galactosidase, cellulase, and xylanase.

12. The method of claim 10, wherein the disorder is selected from the group consisting of: autism; inflammatory bowel disease; Crohn's disease; irritable bowel syndrome; and ulcerative colitis.

13. A method for the treatment of a disorder characterized by a deficiency of isomaltase in a subject, comprising administering to the subject a composition comprising: transglucosidase, glucoamylase (EC 3.2.1.3), maltase, lactase (EC 3.2.1.23), invertase (EC 3.2.1.26), amylase, and a peptidase.

14. The method of claim 13, wherein the peptidase is dipeptidylpeptidase IV.

15. The method of claim 13, wherein the disorder is selected from the group consisting of: autism; inflammatory bowel disease; Crohn's disease; irritable bowel syndrome; and ulcerative colitis.
